(19) United States
(12) Patent Application Publication
(10) Pub. No.: US 2023/0390378 A1
(43) Pub. Date: Dec. 7, 2023

(54) IMMUNOGENIC FORMULATION CONTAINING A MODIFIED BCG STRAIN EXPRESSING AN ANDESVIRUS PROTEIN (ANDV) USEFUL FOR PREVENTING AND TREATING HANTA-ANDV VIRUS INFECTIONS

(71) Applicant: PONTIFICIA UNIVERSIDAD CATOLICA DE CHILE, Santiago (CL)

(72) Inventors: Alexis KALERGIS PARRA, Santiago (CL); Susan BUENO RAMÍREZ, Santiago (CL); Pablo GONZALEZ MUÑOZ, Santiago (CL)

(21) Appl. No.: 18/247,824

(22) PCT Filed: Nov. 10, 2021
(86) PCT No.: PCT/CL2021/050107

§ 371 Date: Apr. 4, 2023

(30) Foreign Application Priority Data
Oct. 5, 2020 (CL) 2568-2020

(51) Int. Cl. A61K 39/12; C07K 14/005; A61P 31/14

(52) U.S. Cl. CPC A61K 39/12; C07K 14/005; A61P 31/14; A61K 2039/522

(57) ABSTRACT

The invention relates to an immunogenic formulation containing the bacillus Calmette-Guerin (BCG) strain in a concentration between 10⁴-10⁹ bacteria, expressing at least one protein or immunogenic fragment of Andesvirus (ANDV), in a pharmaceutically acceptable saline buffer solution, which serves to prepare a vaccine useful for preventing, treating or attenuating ANDV infections. ANDV belongs to the Hantavirus family and is a highly virulent human pathogen, which annually affects dozens of people in Chile generating in some infected a Hantavirus Cardiopulmonary Syndrome (HCPS).

### FIELD OF INVENTION

An immunogenic formulation useful for preparing a vaccine against Andesvirus (ANDV) is reported, where this formulation comprises at least one attenuated strain of Mycobacterium bovis, Bacillus Calmette-Guérin (BCG), which recombinantly expresses one or more proteins or immunogenic fragments of ANDV.

### BACKGROUND OF THE INVENTION

Hantaviruses are the etiological agent of two diseases in humans: hemorrhagic fever with renal syndrome (HFRS) and hantavirus cardiopulmonary syndrome (HCPS). These viruses of worldwide distribution are classified as old world hantaviruses responsible for HFRS, predominate in Europe and Asia the species: Hantaan virus (HTNV) and Seoul virus (SEOV), among others. On the other hand, the hantaviruses of the new world produce HCPS and includes 36 species, among them: Andesvirus (ANDV), Sin nombre (SNV) and Choclo virus (CHOV) which are distributed in America.

The species ANDV is endemic to America and has a high virulence with respect to other hantaviruses, to date it is the only species capable of transmitting from human to human. It is worth mentioning that the development of vaccines against old world hantavirus species is at the forefront, however, these do not offer protection against the species of the new world.

ANDV is an antisense or negative RNA virus comprising in its genome a small segment (S) encoding the nucleocapsid protein (N), a medium segment (M) encoding a precursor glycoprotein that originates the glycoproteins Gn and Gc, and a large segment (L) encoding RNA-dependent polymerase.

Currently, there is no prophylactic treatment capable of generating immunological memory in the host against ANDV infection.

In the state of the art we have found some documents that disclose or protect vaccines against ANDV, among which are WO2019178286A1, CL201802306 and CL201101085, where all these documents use the coated glycoproteins of ANDV, Gn and Gc, as immunogenic particles. These proteins are the most obvious target for a vaccine since being in the capsid is considered to be more exposed to recognition by the host's immune system.

On the other hand, D. M. Custer et al. (Journal of Virology, 2003, vol. 77, no 18, p. 9894-9905) discloses a DNA vaccine comprising the genomic segment M of ANDV, which, as already indicated, also codes for Gn and Gc. Although this strategy is promising, so far there is no legislation or sanitary authorization for the inoculation of foreign DNA in a human host.

Given this scenario, the inventors have developed a new vaccine, with a different strategy to those developed by other groups, which is based mainly on the N protein of ANDV, not on Gn and Gc, and which uses as a vector the attenuated strain of Mycobacterium bovis, Bacillus Calmette-Guérin (BCG), which has demonstrated safe use in neonates for more than 100 years and is known to act as an adjuvant and induce responses that confer long-term immunity. Surprisingly, the inventors have shown that by using the nucleocapsid protein of the virus (N), immunity is generated that allows for control of infection by ANDV.

### DESCRIPTION OF THE FIGURES

FIG. 1. rBCG-N-ANDV expresses recombinant N-ANDV. A specific monoclonal antibody against N-ANDV was used as the primary antibody, followed by a secondary antibody bound to radish peroxidase (HRP). Colonies 2 and 3 (C2 FT and C3 FT) correspond to colony lysates rBCG-N-ANDV. A positive control (recombinant N-ANDV expressed in E. coli, N-ANDV) and negative controls (lysate of rBCG-P-hMPV, BL21-P and water, H₂O) are also shown.

FIG. 2. Animals vaccinated with rBCG-N-ANDV have weight gain. BALB/c male mice 4-6 weeks of age were vaccinated with BCG-WT or rBCG-N-ANDV, or received vehicle (PBS) on days 0 and 14 (booster). Weight measurements were taken several times a week during the 28-day duration of the study. A. Relative change in percentages (%) of weight from day 0. B. Absolute weight (in grams) from day 0. Data is represented as mean+SEM from three animals per group.

FIG. 3. Mice immunized with the rBCG-N-ANDV vaccine have a similar clinical score compared to mice vaccinated with BCG WT. BALB/c male mice 4-6 weeks of age were vaccinated with BCG-WT or rBCG-N-ANDV, or received vehicle (PBS) on days 0 and 14 (booster). Clinical measurements were performed several times a week during the 28-day duration of the study. A. Complete clinical score, covering physiological constants, behavior, appearance, and adverse reactions to immunization, with each category receiving scores from 0 to 3. Specific exposure to adverse reaction to immunization, from 0 to 3. Data is represented as mean+SEM from three animals per group.

FIG. 4. The rBCG-N-ANDV vaccine generates populations of antigen-specific T lymphocytes. BALB/c male mice 4-6 weeks of age were vaccinated with BCG-WT or rBCG-N-ANDV, or received vehicle (PBS) on days 0 and 14 (booster). At day 28, the animals were euthanized and spleen cells were obtained to generate ex vivo stimulation assays with recombinant N-ANDV protein (10 μg/mL and 2 μg/mL) and PPD-B (20 μg/mL). After 72 h of incubation, flow cytometry analyses were performed on spleen cells with various markers. A-C: Increase in CD4+T lymphocytes expressing CD69 and CD71 activation markers. D-F: Increase in CD8+T lymphocytes expressing CD25, CD69, and CD71 activation markers. Data represented as mean f MS obtained from 3 mice per group. **p<0.01, 2-way ANOVA followed by Tukey's multiple comparisons test.

FIG. 5. rBCG-N-ANDV induces proliferation of spleen cells from immunized mice after re-stimulation with N-ANDV. BALB/c male mice 4-6 weeks of age were vaccinated with BCG-WT or rBCG-N-ANDV, or received vehicle (PBS) on days 0 and 14 (booster). At day 28, the animals were euthanized and spleen cells were obtained to generate ex vivo stimulation assays with recombinant N-ANDV protein (10 μg/mL and 2 μg/mL) and PPD-B (20 μg/mL). The spleen cell culture agents obtained at 72 h post-incubation and soluble factor levels were determined by ELISA. Data represented as mean f SEM obtained from 3 mice per group. p<0.0001, 2-way ANOVA followed by Tukey's multiple comparisons test.

### DESCRIPTION OF THE INVENTION

The invention corresponds to a live attenuated vaccine based on Mycobacterium bovis, preferably of the bacillus Calmette-Guérin (BCG) strain that generates the recombinant expression of a heterologous viral antigen, especially the Nucleoprotein (N) of Andesvirus (ANDV) or its immunogenic fragments.

Specifically the invention aims at an immunogenic formulation that confers protection against New World Hantavirus and/or the development of Hantavirus cardiopulmonary syndrome (HCPS), comprising attenuated recombinant strain of Mycobacterium bovis Bacillus Calmette-Guérin (BCG), in an amount between 10⁴-10⁹ colony forming units (CFU) per dose, which expresses at least one protein or immunogenic fragment of Andesvirus (ANDV), in a pharmaceutically acceptable saline buffer solution. Preferably, the protein or immunogenic fragment of Andesvirus corresponds to the N protein of ANDV or its immunogenic fragments, where the protein has an amino acid sequence at least 75% identical to the amino acid sequence of SEQ ID NO:1 and is preferably at least 95% identical to SEQ ID NO: 1.

In the immunogenic formulation of the invention the genes encoding for the N protein of ANDV or its immunogenic fragments are inserted in the genome of Mycobacterium BCG or in extrachromosomal plasmids, in one or more copies, and are regulated or commanded by endogenous or exogenous promoters of Mycobacterium BCG, constitutive or inducible. The genes encoding the N protein of ANDV or its immunogenic fragments correspond to a nucleotide sequence at least 75% identical to SEQ ID NO: 2, and preferably at least 95% identical to SEQ ID NO: 2. As a result, the N protein of ANDV or its immunogenic fragments can be expressed by BCG in a soluble-cytoplasmic way, secreted extracellularly, or as cell membrane-bound proteins. The BCG strain used is preferably chosen between BCG Danish or BCG Pasteur.

In one embodiment of the invention, the protein or immunogenic fragment of Andesvirus amino acid sequences correspond with at least 75%, 80%, 85%, 90%, 95% or more identity with respect to the sequence of the N protein of ANDV, defined according to SEQ ID NO:1, wherein the difference includes substitutions, deletions, additions or insertions. Likewise, for the expert in the art it will be evident that to obtain this protein expression the bacterium must be transformed with a vector containing a nucleotide sequence that encodes for said protein, where the invention includes a nucleotidic sequence with at least 75%, 80%, 85%, 90%, 95% or more of identity with respect to the nucleotide sequence, defined according to SEQ ID NO:2 and its equivalents resulting from the degeneration of the genetic code.

When formulated, the immunogenic formulation of the invention is stabilized by freezing, freeze-drying or saline buffer and excipients for injectable preparations, for preservation prior to use. Among the appropriate excipients are: sodium glutamate, magnesium sulfate heptahydrate, potassium hydrogen phosphate, citric acid monohydrate, L-asparagine monohydrate, iron ammonium citrate, glycerol, and water for injectable preparations and any other available in the art, at the time of execution of the invention.

In another embodiment of the invention protects the use of the immunogenic formulation described to prepare a vaccine to prevent, treat, or attenuate infections of ANDV and the development of Hantavirus cardiopulmonary syndrome (HCPS), where said formulation contains between 1×10⁴-1×10⁹ CFUs (colony forming units) of the recombinant attenuated strain of Mycobacterium BCG stabilized with a saline solution. This vaccine can be administered subcutaneously, percutaneously or subdermally in physiologically acceptable saline.

To generate the formulation that is protected in this invention, the Danish or Pasteur strain of BCG is used, and we transform it at the genome level, expressing the N protein of ANDV constitutively during its replicative cycle. The synthesis of this protein does not generate alterations or impediments in the replicative capacity of the bacterium, so it would not exert a toxicity effect to the vector strain.

Two characteristics of this invention to highlight correspond to its safety profile and immunogenicity, which we have evaluated in a murine preclinical model. Our results of growth curves and clinical manifestations in immunized mice indicate that the administration of this vaccine does not generate significant adverse reactions, having an effect similar to that of untransformed BCG or WT (wild type). With respect to the immunogenicity of this vaccine, we have observed that it generates a proliferation and activation of CD4+ and CD8+ specific T cells against the purified antigen N-ANDV, essential stimuli to produce an immune response and generate immunological memory.

The safety and immunogenicity profile observed for the vaccine of the present invention make it a safe immunization tool.

There is currently no effective vaccine or treatment approved for the prevention or treatment of ANDV infection. The recombinant BCG vaccine expressing the N protein of ANDV (rBCG-N-ANDV) according to the present invention can be used in individuals of all ages. The invention consists of a vaccine developed in order to prevent hantavirus cardiopulmonary syndrome (HCPS). BCG is a good vehicle for vaccine development, as it has been shown to be safe in neonates, children and adults. It can be easily produced on a large scale with low costs and is stable to temperature changes. In addition to this, BCG acts as an adjuvant and induces a Th1 response (T helper lymphocyte), which is necessary for the elimination of the virus. Unlike other formulations, the BCG vector, where the viral protein is expressed, has been widely used for nearly 100 years in humans. In addition, the N protein of ANDV is highly conserved in all subtypes of hantavirus of the new world (36 species), so this vaccine could confer protection against the development of hantavirus cardiopulmonary syndrome throughout the Americas.

As a summary of results achieved by the present invention, we can highlight:
The administration of the rBCG-N-ANDV vaccine in BALB/c model is safe for immunized animals.
Immunization with the rBCG-N-ANDV vaccine generates populations of T lymphocytes in a specific way, so it is immunogenic at the cellular level.

The vaccines of the invention contain live attenuated recombinant strains of Mycobacterium bovis, preferably Bacillus Calmette-Guérin (BCG), for example the BCG Danish or Pasteur strains expressing recombinantly or heterologously one or more proteins or immunogenic fragments ANDV, especially the N protein or its immunogenic fragments. The vaccines of the invention comprise between 1×10⁴-1×10⁹ CFU (colony forming units) of the strains described by doses, and can be kept preserved, prior to administration, in lyophilized form or in a saline solution and stabilizer excipients in cold.

Examples of appropriate stabilizing solutions for immunogenic formulations or vaccines of the invention are:
Diluted Sauton SSI solution (125 μg MgSO 4, 125 μg K₂HPO 4, 1 mg L-asparragine, 12.5 μg ferric ammonium citrate, 18.4 mg 85% glycerol, 0.5 mg citric acid, in 1 ml of H₂O) at 4° C.;
PBS (137 mM NaCl; 2.7 mM KCl; 4.3 mM Na₂HPO₄; 1.47 mM KH₂PO₄, pH 7.4) supplemented with Tween 80 to 0.02% and Glycerol 20% at −80° C.; or
Volume solution: lactose volume 25% and Proskauer and Beck medium supplemented with glucose and Tween 80 (PBGT: 0.5 g asparragine; 5.0 g monopotassium phosphate; 1.5 g magnesium citrate; 0.5 g potassium sulfate; 0.5 ml Tween 80 and 10.0 g glucose per liter of distilled water) lyophilized and preserved in the temperature range between 4° C. and 25° C.

To obtain the recombinant strains of the invention, the genes encoding the N protein or its immunogenic fragments, are inserted into a plasmid, which is incorporated into the bacterium by any available technique. In one embodiment, the plasmid pMV361 is used, incorporated into the bacterium by electrotransformation, and integrated into the bacterial genome by the action of mycobacteriofagos integrases. These genes can also be inserted into extrachromosomal plasmids, such as pMV261, which is incorporated into Mycobacterium by electrotransformation, and maintained extrachromorally in the bacterium. These genes can be in one or several copies, and their expression is commanded by endogenous BCG promoters, constitutive or inducible, for example the promoter of the hsp60 gene and the promoter of the acr gene respectively. These proteins, or immunogenic fragments of ANDV, can be expressed by BCG or other attenuated strains of Mycobacterium, in a soluble-cytoplasmic form, secreted extracellularly, or as membrane-bound proteins.

The amino acid sequence of the N protein of ANDV is found in SEQ ID No.1, and the nucleotide sequence of the gene encoding this N protein of ANDV is found in SEQ ID NO. 2:

### EXAMPLES

These examples are illustrative only and are not intended to limit the range of production or application of the invention. Although specific terms are used in the following descriptions, their use is only descriptive and not limiting.

### Example I: Recombinant BCG Danish Strain for the N Gene of ANDV

The BCG Danish strain was transformed by electrotransformation with the plasmid pMV361/N, derived from the plasmid pMV361, which integrates only once into the genome of the bacterium. This plasmid contains the SEQ ID NO. 1 gene, encoding the N protein of ANDV, SEQ ID NO. 2, which is expressed under the control of the endogenous promoter and constitutive of the BCG gene hsp60. The resulting recombinant colonies were grown (at 37° C. in culture medium Middlebrock 7H9 supplemented, with Kanamycin 25 ug/mL as the antibiotic of choice) up to OD_{600 nm}=1, were centrifuged at 11,000 rpm for 20 min (eppendorf rotor model 5702/R A-4-38) and resuspended in PBS solution (137 mM NaCl; 2.7 mM KCl; 4.3 mM Na₂HPO₄; 1.47 mM KH₂PO₄, pH 7.4), generating doses of 10⁸ CFU per 100 μl and stored at −80° C. until use as a vaccine in animals. The presence of contaminants was ruled out by seeding some of the doses in LB and 7H9 plates without antibiotics. Likewise, it was confirmed that the doses were accurate with respect to the number of CFUs, performing serial dilutions in 7H9 plates and colony counting, from the dose vials.

A fraction of recombinant BCGs was subjected to a protein extraction protocol to evaluate the presence of recombinant N antigen in it. For this, these BCGs were resuspended in a lysis buffer (Tris 50 mM, EDTA 5 mM, SDS 0.6%, protease inhibitor cocktail 1×), subjected to ultrasound pulses (20 pulses of 20 seconds, with a rest stage of 5 minutes every 10 pulses) and then frozen at −20° C. until use in Western blot.

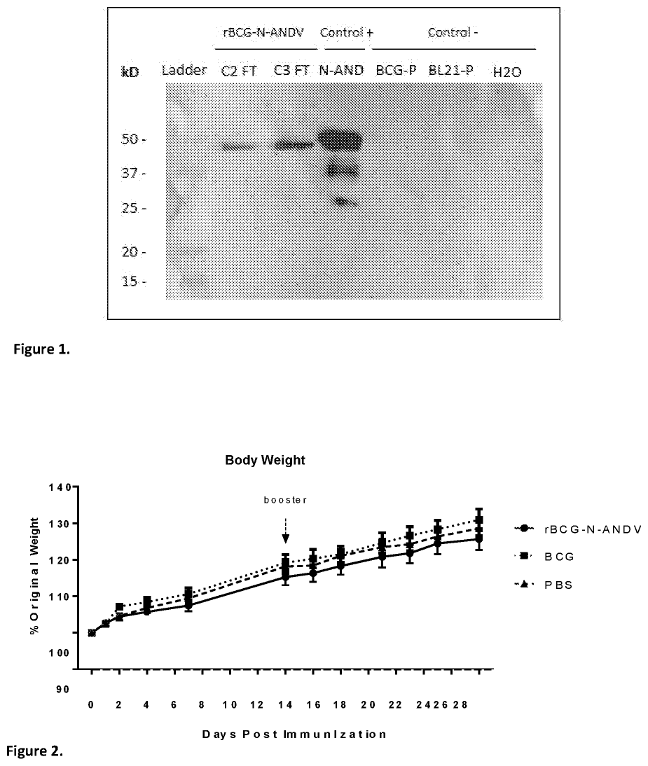

By Western blot, using monoclonal antibodies specific to the N protein of ANDV, the inventors observed that this transformed BCG strain expresses in a way that recombines the N protein of ANDV in the cytoplasm. The results are shown in FIG. 1, where the expression of the N protein of ANDV is observed in 2 colonies of rBCG-N-ANDV, called C2 FT and C3 FT.

### Example II: Clinical Evaluation of Animals Vaccinated with a Formulation of the Recombinant BCG Danish Strain for the N Gene of ANDV

Male BALB/c mice 4-6 weeks of age were vaccinated with the formulation of the invention, the animals received a subcutaneous injection in the neck with 1×10⁸ CFU of rBCG-N-ANDV, or with the untransformed strain BCG-WT or received only vehicle (PBS) at days 0 and 14 of the trial. Weight measurements were taken several times a week during the 28-day duration of the study. The results of this trial are shown in FIG. 2, where we can highlight that animals vaccinated with rBCG-N-ANDV presented a weight gain according to their age and did not present problems in weight gain.

The weight gain of the group that received the formulation of the invention is less than the unimmunized control group (FIG. 2A) and the group immunized with BCG, however, the absolute weight of these animals is similar to the animals of the control groups (FIG. 2B).

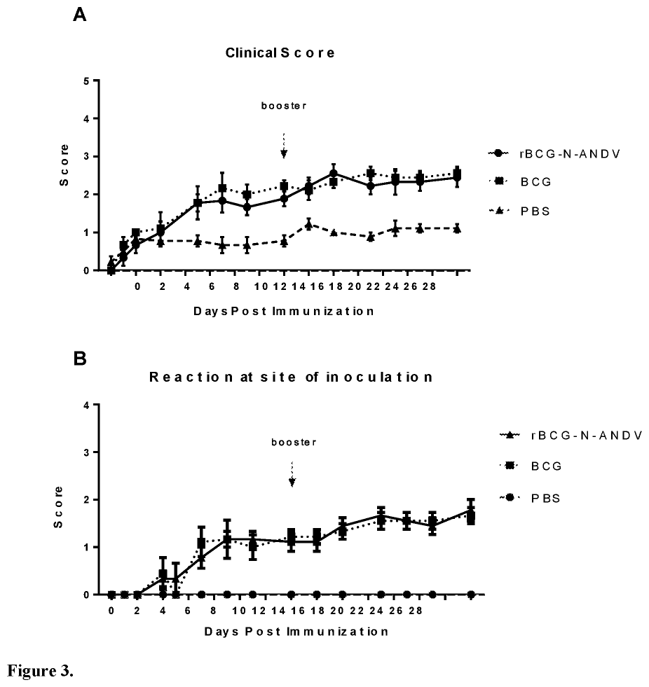

In addition to weight gain, a complete clinical score was studied for each animal where physiological constants, behavior, appearance, and adverse reactions to immunization were evaluated, with each category receiving a score from 0 to 3, where the worst condition would obtain a maximum of 12 points and the best condition 0 points. The results show that the animals immunized with rBCG-N-ANDV did not present physiological or behavioral alterations, with a physiological score that did not exceed 3 points, similar to the control group immunized with BCG without transforming (FIG. 3A).

Finally, the development of reactions at the inoculation site was evaluated, we observed that all animals immunized with rBCG-N-ANDV presented formation of a pseudo-granuloma, which began to appear from the fifth (5th) day after immunization (FIG. 3B). This pseudo-granuloma was accompanied by alopecic areas in the epidermis, without the appearance of erythema, edema or tissue disruption. The animals showed no signs of systemic alterations from this reaction. On the other hand, the animals of the BCG-WT control group also developed pseudo-granuloma, which was evidenced from the seventh (7th) day after inoculation (FIG. 3B). In this group, only one animal presented areas of alopecia, without major local or systemic commitment. We can conclude from this data, that the rBCG-N-ANDV vaccine in high doses is safe and does not generate adverse reactions compared to BCG WT, in this immunization schedule in BALB/c mice.

### Example II: Immunogenicity Evaluation of the Formulation Comprising rBCG-N-ANDV

To evaluate the immunogenicity of the vaccine of the invention male BALB/c mice 4-6 weeks of age were vaccinated with BCG-WT or rBCG-N-ANDV at doses of 1×10⁸ CFU, or received vehicle (PBS) at days 0 and 14 (booster). At day 28, animals were euthanized and spleen cells were obtained to generate ex vivo stimulation assays with recombinant N-ANDV protein (10 μg/mL and 2 μg/mL) and Mycobacterium (20 μg/mL) as a specificity control. After 72 h of incubation, flow cytometry analyses were performed in spleen cells with various markers.

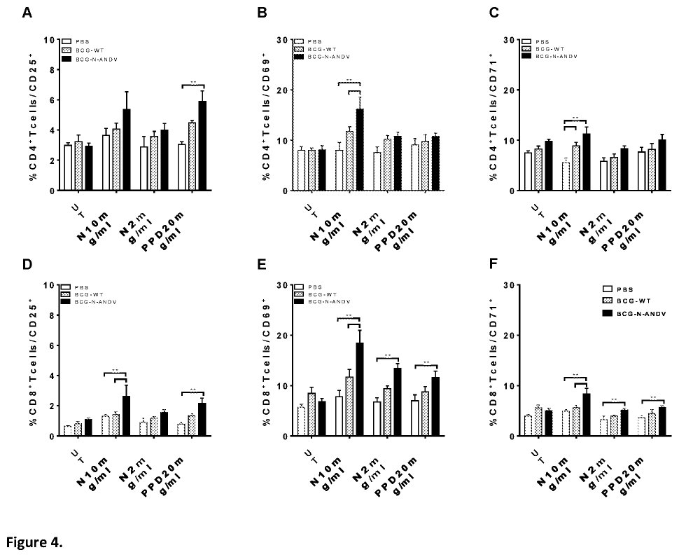

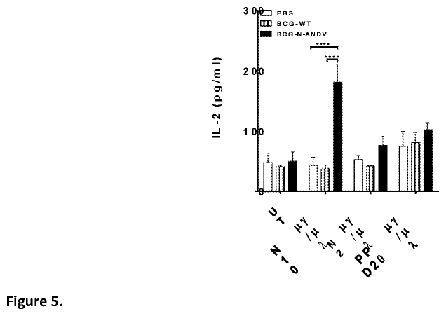

The results suggest that immunization with rBCG-N-ANDV generated CD4+ and CD8+ antigen specific cell populations, which were activated in the presence of the N-ANDV antigen of interest, expressing activation markers such as CD69 (FIGS. 4B and 4E) and CD71 (FIGS. 4C and 4F). Significant differences were also found in IL-2 receptor expression in CD8+ T cells when compared with control groups. The expression of IL-2 measured by ELISA, shows a greater secretion of this interleukin in spleen cells derived from mice immunized with rBCG-N-ANDV when stimulated with recombinant protein N-ANDV, when compared with control groups (FIG. 5), which indicates that there is a specific antigen cell proliferation.

These results demonstrate that animals immunized with rBCG-N-ANDV develop immunity against the N-ANDV protein, which provides protection against Hantavirus, specifically against Andesvirus (ANDV), thus allowing the prevention of the development of Hantavirus Cardiopulmonary Syndrome (HCPS).

1. Immunogenic formulation conferring protection against Hantavirus and/or the development of Hantavirus cardiopulmonary syndrome (HCPS) wherein it comprises an attenuated recombinant strain of Mycobacterium Bacillus Calmette-Guérin (BCG), in an amount between 10⁴-10⁹ CFU per dose, which expresses at least one protein or immunogenic fragment of virus Andesvirus (NVD), in a pharmaceutically acceptable saline buffer solution.

2. The immunogenic formulation according to claim 1, wherein the protein or immunogenic fragment of virus Andesvirus corresponds to the N protein of ANDV or its immunogenic fragments.

3. The immunogenic formulation according to claim 2, wherein the protein or immunogenic fragment of virus Andesvirus corresponds to a sequence of amino acids at least 75% identical to the amino acid sequence of SEQ ID NO: 1.

4. The immunogenic formulation according to claim 3, wherein the immunogenic protein or fragment of Andesvirus virus is at least 95% identical to the sequence SEQ ID NO: 1.

5. The immunogenic formulation according to claim 2, wherein the genes coding for the N protein of ANDV or its immunogenic fragments are inserted in the genome of Mycobacterium BCG or in extrachromosomal plasmids, in one or more copies.

6. The immunogenic formulation according to claim 5, wherein the genes encoding the N protein of ANDV or its immunogenic fragments correspond to a nucleotide sequence at least 75% identical to SEQ ID NO: 2.

7. The immunogenic formulation according to claim 6, wherein the genes encoding the N protein of ANDV or its immunogenic fragments correspond to a nucleotide sequence at least 95% identical to SEQ ID NO: 2.

8. The immunogenic formulation according to claim 7, wherein the expression of genes are commanded by endogenous or exogenous promoters of Mycobacterium BCG, either constitutive or inducible.

9. The immunogenic formulation according to claim 8, wherein the N protein of ANDV or its immunogenic fragments can be expressed by BCG in a soluble-cytoplasmic way, secreted extracellularly or as proteins bound to cell membrane.

10. The immunogenic formulation according to claim 1, wherein it is stabilized by freezing, freeze-drying or saline buffer and excipients for preservation prior to use.

11. The immunogenic formulation according to claim 9, wherein it is stabilized by freezing, freeze-drying or saline buffer and excipients for preservation prior to use.

12. The immunogenic formulation according to claim 1, wherein the attenuated recombinant strain is BCG Danish or BCG Pasteur.

13. Use of the immunogenic formulation according to claim 1 wherein it serves to prepare a vaccine to prevent, treat or attenuate ANDV infections and/or the development of Hantavirus cardiopulmonary syndrome (HCPS), wherein said formulation contains between 1×10⁴-1×10⁹ colony-forming units of the recombinant attenuated strain of Mycobacterium BCG stabilized with physiologically acceptable saline solution.

14. Use according to claim 13, wherein it serves to prepare a vaccine to prevent, treat or attenuate infections of ANDV and/or the development of Hantavirus cardiopulmonary syndrome (HCPS), to be administered subcutaneously, percutaneously or subdermally in a physiologically acceptable saline solution.


SEQUENCE LISTING (2 sequences)

SEQ ID NO: 1 - protein, 428 aa, Andes virus
MSTLQELQENITAHEQQLVTARQKLKDAEKAVEVDPDDVNKSTLQNRRAAVSTLETKLGELKRQLADLVAAQKLATKPVDPTGLEPDDHLKEKSSLRYGNVLDVNSIDLEEPSGQTADWKAIGAYILGFAIPIILKALYMLSTRGRQAVKDNKGTRIRFKDDSSFEEVNGIRKPKHLYVSMPTAQSTMKAEEITPGRFRTIACGLFPAQVKARNIISPVMGVIGFGFFVKDWMDRIEEFLAAECPFLPKPKVASEAFMSTNKMYFLNRQRQVNESKVQDIIDLIDHAETESATLFTEIATPHSVWVFACAPDRCPPTALYVAGVPELGAFFSILQDMRNTIMASKSVGTAEEKLKKKSAFYQSYLRRTQSMGIQLDQKIIILYMLSWGKEAVNHFHLGDDMDPELRQLAQSLIDTKVKEISNQEPLKL

SEQ ID NO: 2 - DNA, 1287 bp, Andes virus
atgagcaccctccaagaattacaagaaaacatcacagcacacgaacaacagctcgtgactgctcggcaaaagcttaaagatgccgagaaggcggtggaggtggacccggatgacgttaacaagagcacactacaaaatagacgggcagctgtgtctacattggagaccaaactcggggaactcaagagacaacttgcagatctggtggcagctcaaaaattggctacaaaaccagttgatccaacagggcttgagcctgatgaccatctgaaagagaaatcatctttgagatatgggaatgtcctggatgttaactcaatcgatttggaagaaccgagtggacagactgctgattggaaggctataggagcatatatcttagggtttgcaattccgatcatactaaaagccctatatatgctgtcaacccgtggaagacagactgtgaaagacaacaaaggaaccaggataaggtttaaggatgattcttcctttgaagaggtcaatgggatacgtaaaccgaaacacctttatgtctcaatgccaactgcacaatctactatgaaggctgaggaaattacgccaggacggtttaggacaattgcttgtggcctctttccagcacaggtcaaagctcgaaatataataagtcctgtgatgggagtaatcggatttggcttctttgtgaaggactggatggatcggatagaggaattcctggctgcagagtgtccattcctgcctaagccaaaggttgcctcagaagccttcatgtctaccaacaagatgtattttctgaatagacaaagacaagtcaatgaatctaaggttcaagacatcattgatttaatagatcatgctgaaactgaatctgctaccttgtttacagaaattgcaacaccccattcagtctgggtgtttgcatgtgcgcctgaccggtgccctccgactgcattgtatgttgcaggggtaccagaacttggtgcattcttttccattcttcaggacatgcgcaataccatcatggcatccaaatctgtggggactgcagaagagaagctgaagaagaaatctgccttttatcaatcttacctaagaaggacacaatctatggggattcaactggaccagaaaatcataattctctacatgctttcatggggtaaagaagctgtgaatcatttccatcttggtgatgatatggaccctgaactaaggcagctagctcaatccctgattgacaccaaggtgaaggagatctccaaccaagagccacttaagttgtaa